(19) United States
(12) Patent Application Publication
(10) Pub. No.: US 2006/0052788 A1
(43) Pub. Date: Mar. 9, 2006

(54) Expandable fixation devices for minimally invasive surgery

(76) Inventors: Sarah L. Thelen, North Manchester, IN (US); Antony J. Lozier, Warsaw, IN (US); Nicolas J. Pacelli, Culver, IN (US)

Correspondence Address: ZIMMER TECHNOLOGY - BAKER & DANIELS, 111 EAST WAYNE STREET, SUITE 800, FORT WAYNE, IN, 46802, US

(21) Appl. No.: 11/243,789

(22) Filed: Oct. 5, 2005

Related U.S. Application Data
(63) Continuation-in-part of application No. 11/061,898, filed on Feb. 18, 2005.
Application No. 11/061,898 is a continuation-in-part of application No. 10/358,009, filed on Feb. 4, 2003, now abandoned.
(60) Provisional application No. 60/621,487, filed on Oct. 22, 2004.
(60) Provisional application No. 60/654,481, filed on Feb. 18, 2005.

(51) Int. Cl. A61B 17/58

(52) U.S. Cl. 606/72; 606/151

(57) ABSTRACT

A plurality of expandable fixation devices (EFDs) for minimally invasive surgery. The EFDs use an expanding deformation of the device to achieve a mechanical interference with the environment, e.g., bone. The shape of expansion can be controlled by many factors including the original geometry of the part, the geometry of material removed from the part, and the amount of deformation applied to the part. The EFD may be a relatively large anchor-type fixation device generally characterized by a single set of long longitudinal cuts parallel to a central axis of the device or concentric to a central curve of the device. The EFD may alternatively be a relatively small anchor-type fixation device generally characterized by multiple sets of shorter longitudinal cuts parallel to a central axis of the device or concentric to a central curve of the device. The EFD may also be a screw-type fixation device generally characterized by multiple rotationally indexed cuts in a helical pattern. The cuts define intermediate material regions which form protrusions on the EFD when deformed and, taken together, form a non-continuous helical thread to anchor the EFD in a bone.

### CROSS REFERENCE TO RELATED APPLICATIONS

This application claims the benefit under Title 35, U.S.C. § 119(e) of U.S. Provisional Patent Application Ser. No. 60/621,487, entitled Transformation Technology Hip Fracture Fixation Device and Method for the Treatment of Hip Fractures, filed Oct. 22, 2004, and U.S. Provisional Patent Application Ser. No. 60/654,481, entitled Method and Apparatus for Reducing Femoral Fractures, filed Feb. 18, 2005.

This application is a continuation-in-part of prior co-pending U.S. patent application Ser. No. 11/061,898, filed Feb. 18, 2005, which is a continuation-in-part of prior U.S. patent application Ser. No. 10/358,009, filed Feb. 4, 2003.

This application hereby expressly incorporates by reference herein the entire disclosures of U.S. Provisional Patent Application Ser. No. 60/654,481, filed Feb. 18, 2005; U.S. Provisional Patent Application Ser. No. 60/621,487, filed Oct. 22, 2004; U.S. patent application Ser. No. 11/061,898, filed Feb. 18, 2005; U.S. patent application Ser. No. 10/358,009, filed Feb. 4, 2003; U.S. patent application Ser. No. 10/266,319, filed Oct. 8, 2002; U.S. patent application Ser. No. 10/155,683, filed May 23, 2002; and U.S. patent application Ser. No. 09/520,351, filed Mar. 7, 2000, now U.S. Pat. No. 6,447,514.

### BACKGROUND

1. Field of the Invention

The present invention relates to expandable fixation devices and, more particularly, to expandable fixation devices for use in minimally invasive surgery.

2. Description of the Prior Art

Many procedures in orthopedic surgery require the use of fixation devices. For example, lag screws, plate screws, fragment fixation screws, pedicle screws, and acetabular cup fixation screws are all used as fixation devices in orthopedic surgery. In many applications, an access hole is provided in a bone of a patient which is dimensioned slightly smaller than the outer threads of known fixation devices, such as a typical screw. Typical screws have outer threads which have a larger diameter than the remainder of the body of the screw to ensure engagement of the threads with the bone. Furthermore, in many applications, a bone plate is positioned and then an entire lag screw, including threads, is inserted through a screw hole in the bone plate and engaged with the bone stock.

Alternatively, in hip fracture fixation surgery, a hip screw and plate combination requires an access hole to be drilled into a patient's bone from the lateral cortex to the femoral head of a patient's femur. A lag screw is then inserted into the access hole and threaded into unresected bone surrounding the access hole near the femoral head. The hip plate is then placed into the access hole and along the length of the femur. The hip plate receives the lateral end of the lag screw through an aperture or screw hole therein. The hip plate must be inserted after the lag screw because the outer threads of the lag screw have a diameter greater than the diameter of the aperture in the hip plate. Once inserted into the bone, the hip plate is attached along the length of the femur by a series of plate screws.

Although the foregoing methods have been effective, what is needed are a method and devices for anchoring in a bone which are an improvement over the foregoing.

### SUMMARY

The present invention provides a plurality of expandable fixation devices (EFDs) for minimally invasive surgery. The EFDs of the present invention are expandingly deformable to achieve a mechanical interference with the environment, e.g., bone. The shape of expansion can be controlled by many factors including the original geometry of the part, the geometry of material removed from the part, and the amount of deformation force applied to the part. In one embodiment, the EFD is a relatively large anchor-type fixation device generally characterized by a single set of long longitudinal cuts parallel to a central axis of the device or concentric to a central curve of the device. In an alternative embodiment, the EFD is a relatively small anchor-type fixation device generally characterized by multiple sets of shorter longitudinal cuts parallel to a central axis of the device or concentric to a central curve of the device. In yet another alternative embodiment, the EFD is a screw-type fixation device generally characterized by multiple rotationally indexed cuts in a helical pattern. The screw-type EFD may advantageously be removed by simply unthreading the device. The EFDs of the present invention advantageously allow smaller screw holes in bone plates and allow a hip plate to be inserted and attached to the femur prior to inserting a lag screw.

In one form thereof, the present invention provides an expandable fixation device having a first end and a second end for use in orthopedic surgery for anchoring within an anatomical structure including a body having a central axis and including at least one expandable element, each expandable element defined between a pair of openings in the body, wherein upon compressive loading of the body, each expandable element expands radially with respect to the central axis to anchor the device within the anatomical structure.

In another form thereof, the present invention provides an expandable fixation device having a first end and a second end for use in orthopedic surgery for anchoring within an anatomical structure including a body having a central axis; and at least one set of apertures formed in the body, the apertures circumferentially formed around the body and defining expandable portions between the apertures, at least some of the expandable portions axially and circumferentially staggered around the body, wherein, upon compressive loading of the body, the expandable portions radially expand and, taken together, form at least a portion of a helical thread.

### BRIEF DESCRIPTION OF THE DRAWINGS

The above mentioned and other features and objects of this invention, and the manner of attaining them, will become more apparent and the invention itself will be better understood by reference to the following description of exemplary embodiments of the invention taken in conjunction with the accompanying drawings, wherein:

FIG. 1 is a perspective view of an expandable fixation device according to one embodiment;

FIG. 2 is a cross-sectional view of a portion of the expandable fixation device of FIG. 1, taken along line 2-2 of FIG. 1;

FIG. 3 is a cross-sectional view of the expandable fixation device of FIG. 1;

FIG. 4 is a cross-sectional view of the expandable fixation device of FIG. 1, further illustrating the expansion of the radially expandable fingers;

FIG. 5 is a plan view of an expandable fixation device according to an alternative embodiment;

FIG. 6 is a perspective view of the expandable fixation device of FIG. 5, further illustrating the expansion of the radially expandable fingers;

FIG. 7 is a plan view of an expandable fixation device according to another alternative embodiment, further illustrating a rod actuator;

FIG. 8 is a plan view of the expandable fixation device of FIG. 7, further illustrating the expansion of the radially expandable fingers;

FIG. 9 is a cross-sectional view of the expandable fixation device of FIG. 8;

FIG. 10 is a plan view of an expandable fixation device according to yet another alternative embodiment;

FIG. 11 is a plan view of an expandable fixation device according to a still further alternative embodiment;

FIG. 12 is a plan view of an expandable fixation device according to an alternative embodiment, further illustrating the expansion of the radially expandable fingers;

FIG. 13 is a cross-sectional view of a portion of the expandable fixation device of FIG. 12, further illustrating a rod and plunger deployment device;

FIG. 14 is a cross-sectional view of a portion of the expandable fixation device of FIG. 12, further illustrating the rod and plunger deployment device partially expanding the radially expandable fingers;

FIG. 15 is a cross-sectional view of a portion of the expandable fixation device of FIG. 12, further illustrating the rod and plunger deployment device further expanding the radially expandable fingers;

FIG. 16 is a cross-sectional view of a portion of the expandable fixation device of FIG. 12, further illustrating the substantially complete expansion of the radially expandable fingers and a partial deformation of the deployment device;

FIG. 17 is an exploded perspective view of an expandable fixation device actuator according to one embodiment;

FIG. 18 is a perspective view of the actuator of FIG. 17;

FIG. 19 is an exploded perspective view of an expandable fixation device actuator according to an alternative embodiment;

FIG. 20 is a perspective view of the actuator of FIG. 19;

FIG. 21 is a plan view of an expandable fixation device according to another alternative embodiment;

FIG. 22 is a plan view of the expandable fixation device of FIG. 21, further illustrating expansion of the intermediate material regions;

FIG. 23 is a plan view of an expandable fixation device according to a yet further alternative embodiment;

FIG. 24 is a plan view of the expandable fixation device of FIG. 23, further illustrating expansion of the intermediate material regions;

FIG. 25 is a plan view of an expandable fixation device according to a still further alternative embodiment;

FIG. 26 is a plan view of the expandable fixation device of FIG. 25, further illustrating expansion of the intermediate material regions;

FIG. 27 is a partial plan view of an expandable fixation device according to an alternative embodiment;

FIG. 28 is an end view of the expandable fixation device of FIG. 27, viewed along line 28-28 of FIG. 27;

FIG. 29 is a plan view of the expandable fixation device of FIG. 27, further illustrating expansion of the intermediate material regions;

FIG. 30 is an end view of the expandable fixation device of FIG. 29, viewed along line 30-30 of FIG. 29;

FIG. 31 is a plan view of an expandable fixation device according to another alternative embodiment;

FIG. 32 is a plan view of the expandable fixation device of FIG. 31, further illustrating expansion of the intermediate material regions;

FIG. 33 is a plan view of an expandable fixation device in according to yet another alternative embodiment;

FIG. 34 is a plan view of the expandable fixation device of FIG. 33, further illustrating expansion of the intermediate material regions;

FIG. 35 is a plan view of an expandable fixation device according to a still further alternative embodiment; and

FIG. 36 is a plan view of the expandable fixation device of FIG. 35, further illustrating expansion of the intermediate material regions.

Corresponding reference characters indicate corresponding parts throughout the several views. Although the drawings represent embodiments of the present invention, the drawings are not necessarily to scale and certain features may be exaggerated in order to better illustrate and explain the present invention. The exemplifications set out herein illustrate embodiments of the invention, and such exemplifications are not to be construed as limiting the scope of the invention in any manner.

### DETAILED DESCRIPTION

The embodiments disclosed below are not intended to be exhaustive or limit the invention to the precise forms disclosed in the following detailed description. Rather, the embodiments are chosen and described so that others skilled in the art may utilize their teachings.

The description below may include reference to the following terms: lateral (at or near the left or right side of the body, farther from the midsagittal plane, as opposed to medial); medial (in the middle, at or near the midsagittal plane, as opposed to lateral); proximal (nearest the surgeon, as opposed to distal); and distal (further from the surgeon, as opposed to proximal). In the present application, the terms “lateral” and “medial” are used in the exemplary context of a lag used for a hip fracture reduction surgery, such as that described in U.S. Provisional Patent Application Ser. No. 60/654,481, filed Feb. 18, 2005; U.S. Provisional Patent Application Ser. No. 60/621,487, filed Oct. 22, 2004; U.S. patent application Ser. No. 11/061,898, filed Feb. 18, 2005; U.S. patent application Ser. No. 10/358,009, filed Feb. 4, 2003; U.S. patent application Ser. No. 10/266,319, filed Oct. 8, 2002; U.S. patent application Ser. No. 10/155,683, filed May 23, 2002; and U.S. patent application Ser. No. 09/520,351, filed Mar. 7, 2000, now U.S. Pat. No. 6,447,514, the disclosures of which are expressly incorporated herein by reference. However, the terms “lateral” and “medial”, as used herein, are not meant to be limiting and other terms, such as anterior and posterior, posterior and anterior, superior and inferior, inferior and superior, proximal and distal, and distal and proximal, may be applicable, depending on the specific application of the lag.

The present invention generally provides a plurality of expandable fixation devices (EFDs). In various embodiments, the EFD includes a plurality of radially expandable tines or fingers. In other embodiments, the EFD includes a plurality of longitudinal apertures defining intermediate material regions therebetween which, when deformed, provide fixation. In yet other embodiments, the EFD includes a plurality of longitudinal apertures strategically located and formed to provide a threaded configuration when deformed. The present invention advantageously provides for the capability of threading the EFD out of a bone after implantation, thereby reducing the amount of tissue damage. Additionally, the present invention advantageously provides for a thinner wall construction of the EFD, thereby reducing cost of construction. The EFDs of the present invention may be used as lag screws, compression hip screws, plate screws, fragment fixation screws, acetabular cup fixation screws, pedicle screws, intramedullary nailing systems, and gamma or intertrochanteric-subtrochanteric (ITST) nailing systems, for example. Depending upon the particular design, some the EFDs of the present invention have enhanced pull-out strength, i.e., the axial resistant force which prevents the EFD from being axially pulled out, cut-out strength, i.e., the resistance to the force causing the threads to cut through bone during weight-bearing activity, and fatigue strength, i.e., the overall strength of the device and resistance to mechanical or stress-induced failure, when compared to prior fixation devices.

A. Expandable Fixation Devices

1. Large Anchor-Type Expandable Fixation Devices

The EFDs described in this section are generally characterized by a single set of longitudinal cuts parallel to the central axis or concentric to the central curve of the part. The EFDs typically have one level of expansion. The EFDs are capable of expanding to a size significantly greater than their original geometry, e.g., 75%-100% increase in effective diameter depending on the application. The expanded size is a function of longitudinal cut length and can be varied significantly.

Referring now to FIGS. 1-4, expandable fixation device (EFD) 100 is shown and includes body 104, lateral end 112, medial end 106 including apertures 108 (only one of which is shown) positioned 180° apart, radially expandable tines/fingers or expandable elements 102, and interior surface 148. Lateral end 112 may include insertion notches 114 for coupling of an inserting or retracting instrument (not shown) to insert or retract EFD 100 into or out of a patient. Each radially expandable finger 102 is defined between outer circumferential grooves 130 and between respective pairs of longitudinal cuts 140. Longitudinal cuts 140 may be slots formed in body 104, or, alternatively, may be formed by line cuts formed in body 104 which are substantially longitudinally oriented. Longitudinal cuts 140 are parallel to central axis 101. Outer circumferential grooves 130 and inner circumferential groove 132 create hinge points for radially expandable fingers 102 and facilitate deformation into the position shown in FIG. 4. The central portion of each radially expandable finger 102 may include small central cutouts 129 on one or opposing sides thereof, as shown in FIG. 1. Cutouts 129 further facilitate deformation of radially expandable fingers 102 by defining a localized area of reduced thickness which provides a bending or hinge point. In an alternative embodiment, radially expandable fingers 102 are provided with additional hinge points which allow for radially expandable fingers 102 to be deformed into shapes differing from the triangular shape shown in FIG. 4. For example, in one exemplary embodiment (not shown), four hinge points are provided such that radially expandable fingers 102 form a trapezoidal shape upon deformation.

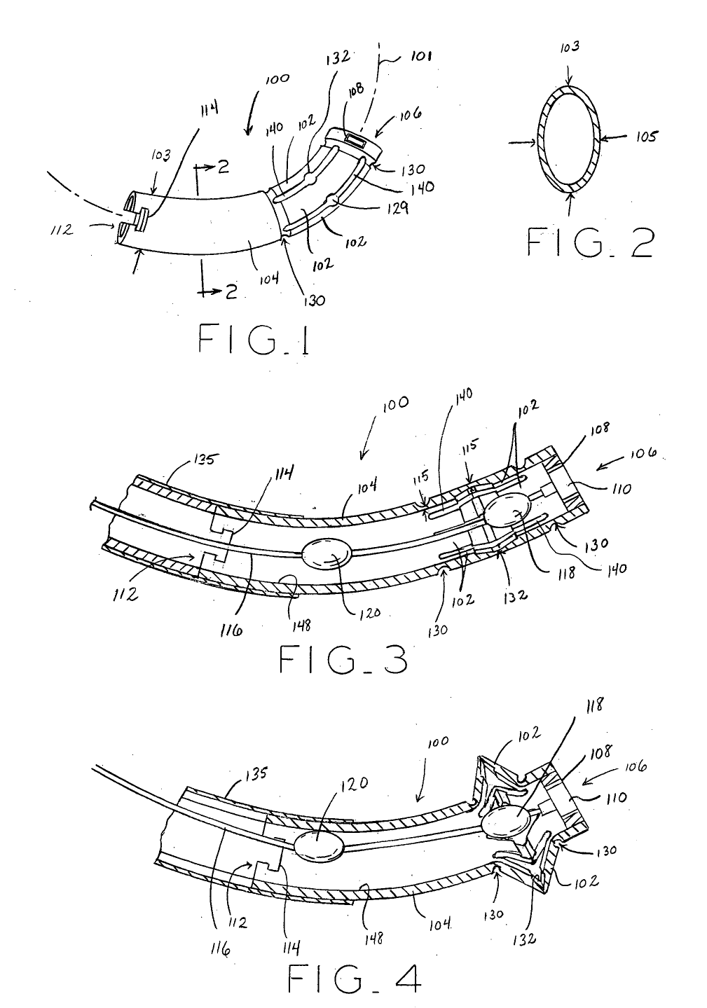

As shown in FIG. 2, EFD 100 may have a substantially elliptical cross-section throughout its entire length with major axis 103 and minor axis 105. The dimensions of EFD 100 may be slightly less than the interior dimensions of a lag tube 135 of a femoral implant, similar to the lag tubes fully described in U.S. Provisional Patent Application Ser. No. 60/654,481, filed Feb. 18, 2005; U.S. Provisional Patent Application Ser. No. 60/621,487, filed Oct. 22, 2004; U.S. patent application Ser. No. 11/061,898, filed Feb. 18, 2005; U.S. patent application Ser. No. 10/358,009, filed Feb. 4, 2003; U.S. patent application Ser. No. 10/266,319, filed Oct. 8, 2002; U.S. patent application Ser. No. 10/155,683, filed May 23, 2002; and U.S. patent application Ser. No. 09/520,351, filed Mar. 7, 2000, now U.S. Pat. No. 6,447,514, the disclosures of which are expressly incorporated by reference herein. In one embodiment, major axis 103 of the elliptical cross-section is coplanar with the arc defined by central axis 101 of EFD 100. Major axis 103 of EFD 100 may be slightly less than the diameter of a reamer head used to form a cavity into which EFD 100 is placed. In one embodiment, major axis 103 is approximately 12.7 mm (0.5 in.) and fingers 102 expand to approximately 25.4 mm (1.0 in.) along major axis 103 and 21.6 mm (0.85 in.) along minor axis 105. EFD 100 may be formed in a plurality of lengths to accommodate a wide range of applications. Alternatively, EFD 100 may have a substantially circular cross-section. In yet another alternative embodiment, EFD 100 may be equipped with reaming features, such as including flats or cutting surfaces on fingers 102.

As shown in FIG. 3, the notched regions near outer circumferential grooves 130 which locate bending points for fingers 102 upon deformation have wall thickness 115. Wall thicknesses 115 which have been found suitable are between about 0.46 mm (0.018 in.) and about 1.02 mm (0.040 in), or preferably about 0.76 mm (0.030 in), but other thicknesses may be used. These thicknesses of wall thickness 115 typically require a deformation force between about 181 kg (400 lbs.) and 363 kg (800 lbs.), or preferably about 227 kg (500 lbs.). A suitable wall thickness of the remaining portions of EFD 100, in one embodiment, is about 1.34 mm (0.053 in.), but other thicknesses may be used. EFD 100 may be constructed of, for example, stainless steel, and be electropolished or glass bead blasted for a matte finish.

Referring now to FIGS. 3, 4, and 17, anchor 110 may be provided to facilitate deformation of radially expandable fingers 102 of EFD 100, as described below. Anchor 110 includes oppositely oriented engagement ears 136 which extend along major axis 103 of EFD 100 upon insertion into EFD 100. Anchor 110 is sized to be inserted from lateral end 112 of EFD 100 and to be engaged with apertures 108 in medial end 106 of EFD 100. Apertures 108 are formed so that a 90° rotation of anchor 110 makes engagement ears 136 perpendicular to major axis 103 and engaged with apertures 108.

Referring still to FIGS. 3, 4, and 17, cable 116 is coupled to anchor 110. Cable 116 is formed of a biocompatible, sterilizable material such as stainless steel or cobalt chrome alloy. Cable 116 may be formed as a single strand or may be formed by braiding two or more strands of wire.

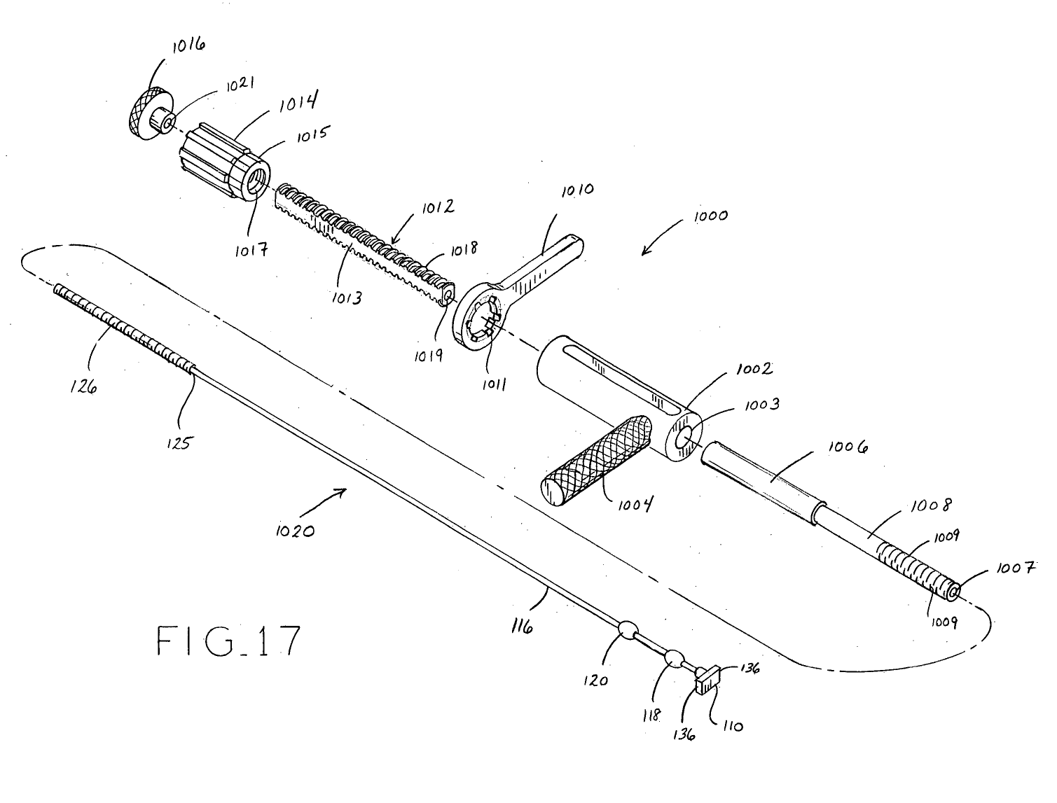

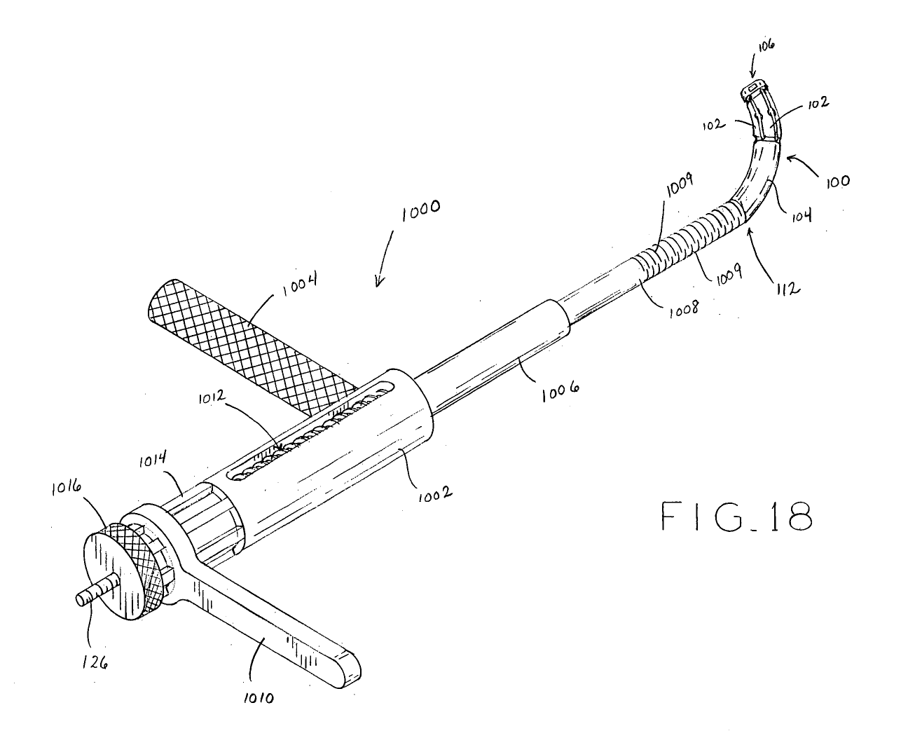

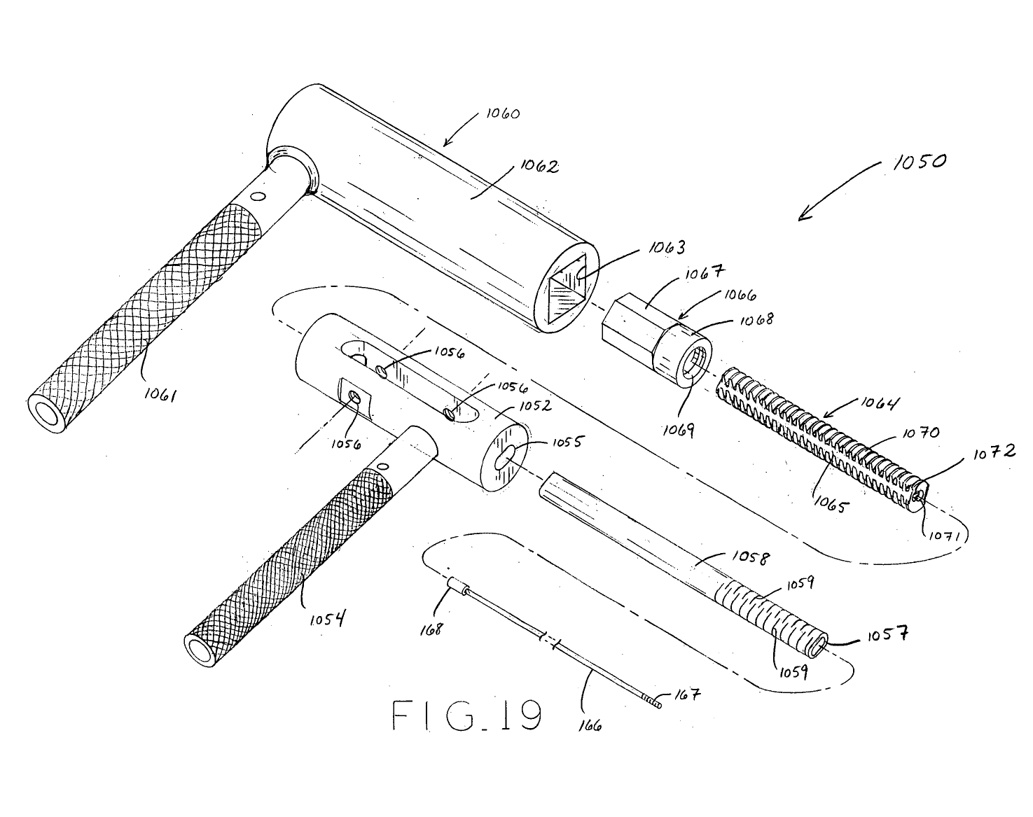

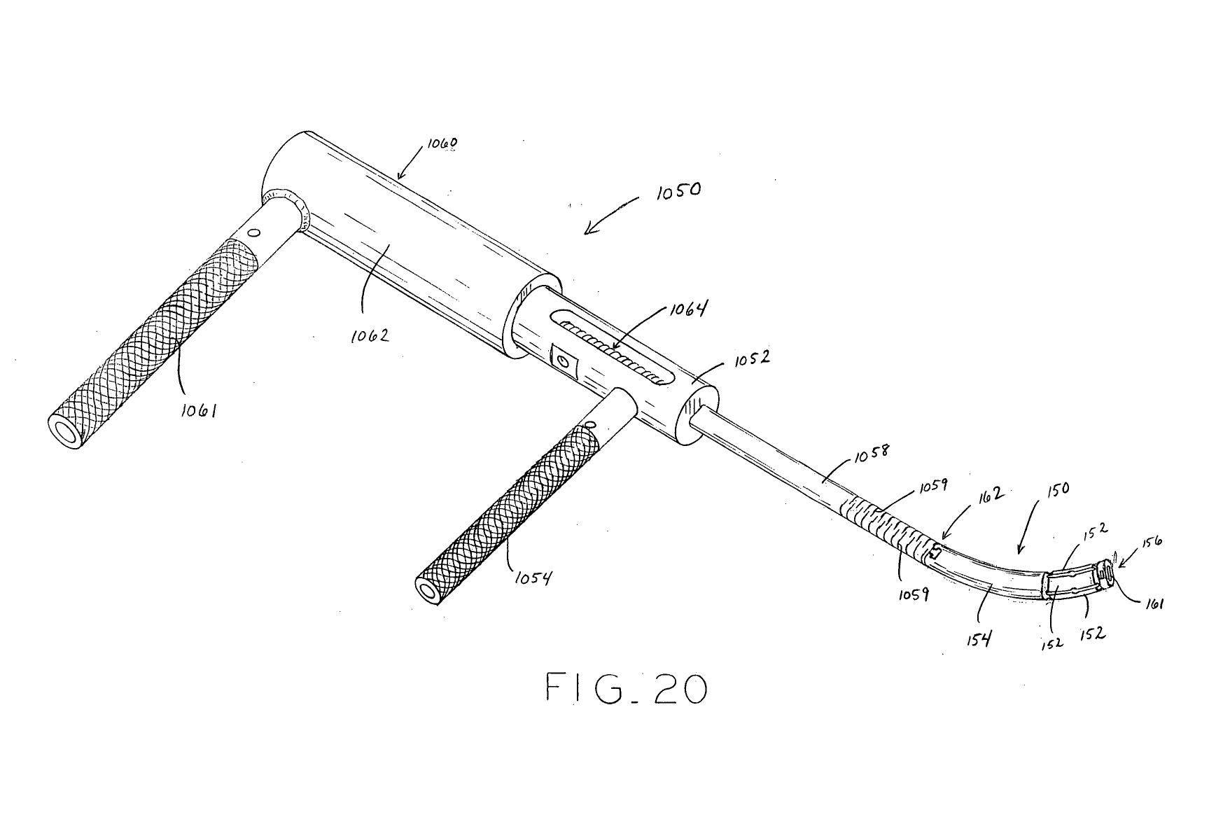

Medial spacer 118 and lateral spacer 120 are cannulated and are strung on cable 116. Medial spacer 118 is held in position on cable 116, for example, by crimped beads (not shown) so that medial spacer 118 is free to rotate on cable 116 and is held in position within EFD 100 adjacent fingers 102. Because of the arcuate shape of EFD 100 and the geometry of fingers 102, finger 102 located on the concave side of EFD 100 may tend to expand inwardly rather than outwardly upon deformation by EFD actuator 1000 (FIGS. 17-18) or EFD actuator 1050 (FIGS. 19-20), described below, or another similar actuator device. If one finger 102 initially folds inward as medial end 106 is compressed toward lateral end 112, the inwardly expanding finger 102 contacts medial spacer 118. Contact with medial spacer 118 forces the inwardly expanding finger 102 to expand outward in conformance with the remainder of radially expandable fingers 102. Lateral spacer 120 facilitates centering of cable 116 in EFD 100 and prevents cable 116 from damaging interior surface 148 of EFD 100 or any device attached thereto.

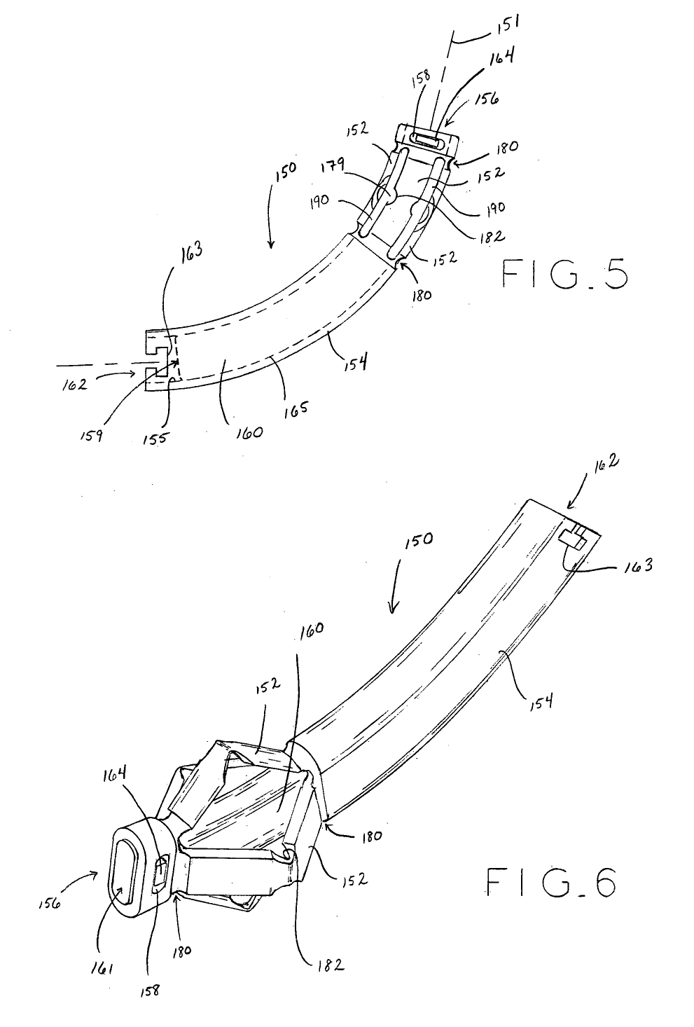

In one alternative embodiment, shown in FIGS. 5-6, EFD 150 is shown which, except as described below, is substantially similar in structure and operation to EFD 100 (FIGS. 1-4) described above. EFD 150 includes body 154, radially expandable fingers or elements 152, interior surface 155, medial end 156 including medial aperture 158, and lateral end 162 including notches 163. Each radially expandable finger 152 is defined between outer circumferential grooves 180 and between respective pairs of longitudinal cuts 190. Longitudinal cuts 190 are parallel to central axis 151. Outer circumferential grooves 180 and inner circumferential groove 182 create hinge points for radially expandable fingers 152 and facilitate deformation into the position shown in FIG. 6 by defining localized areas of reduced wall thickness. As shown in FIG. 5, the central portion of each radially expandable finger 152 may include small central cutouts 179 on one or opposing sides thereof. Cutouts 179 further facilitate deformation of radially expandable fingers 152 by defining a localized area of reduced thickness which provides a bending or hinge point.

Referring still to FIGS. 5-6, in one embodiment, EFD 150 includes EFD support 160 which provides support to body 154 of EFD 150 and facilitates prevention of inward expansion of fingers 152. EFD support 160 includes lateral end 159 and medial end 161. Medial end 161 includes an aperture (not shown) for accepting transverse pin 164, as described below. Lateral end 159 may include a threaded recess (not shown) for coupling to an insertion and actuation instrument, as described below. EFD support 160 is arcuate and shaped to match the curve of EFD 150. Exterior surface 165 of EFD support 160 substantially matches with interior surface 155 of EFD 150 throughout the entire length of EFD support 160. EFD support 160 may be made of a suitable material, for example, 316L stainless steel. EFD support 160 is inserted into EFD support 150 through lateral end 162 and secured therein with transverse pin 164. Transverse pin 164 traverses aperture 158 in EFD 150 and a corresponding aperture (not shown) provided in the medial end of EFD support 160 and forms a press-fit engagement with the aperture in EFD support 160. Transverse pin 164 is dimensioned to extend beyond both sides of EFD support 160 to provide a locking engagement between EFD 150 and EFD support 160. The press-fit engagement prevents accidental removal of transverse pin 164 and the consequential disengagement of EFD support 160 from EFD 150. In one embodiment, medial end 161 of EFD support 160 is substantially flush with medial end 156 of EFD 150.

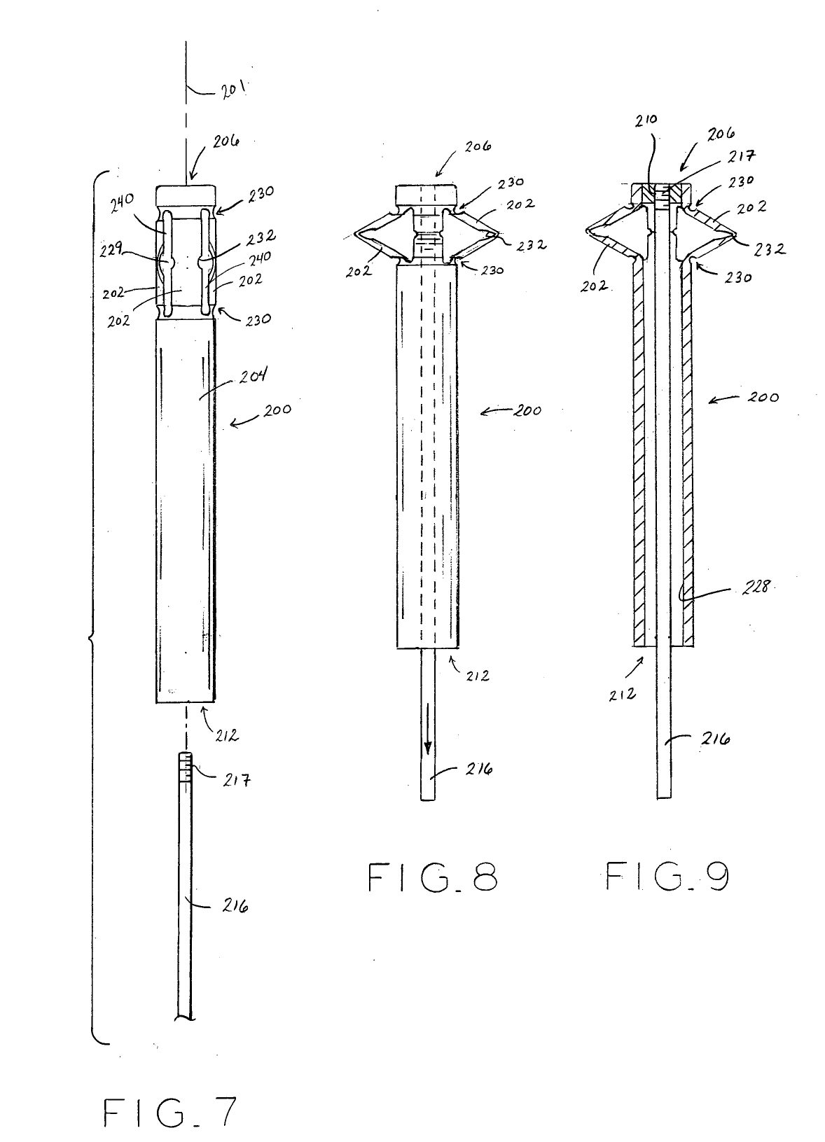

Referring now to FIGS. 7-9, in one alternative embodiment, EFD 200 is shown which, except as described below, is substantially similar in structure and operation to EFD 150 (FIGS. 5-6) described above. EFD 200 includes body 204, radially expandable fingers or elements 202, interior surface 228, medial end 206, and lateral end 212. Each radially expandable finger 202 is defined between outer circumferential grooves 230 and between respective pairs of longitudinal cuts 240. Longitudinal cuts 240 are parallel to central axis 201. Outer circumferential grooves 230 and inner circumferential groove 232 create hinge points for radially expandable fingers 202 and facilitate deformation into the position shown in FIGS. 8-9. The central portion of each radially expandable finger 202 may include small central cutouts 229 on one or opposing sides thereof, as shown in FIG. 7. Cutouts 229 further facilitate deformation of radially expandable fingers 202 by defining a localized area of reduced thickness which provides a bending or hinge point.

Medial end 206 may include threaded recess 210 therein into which threaded end 217 of EFD rod 216 can be threaded. In operation, EFD rod 216 may be substantially similar in function to EFD cable 166 (FIG. 19), described below, but EFD rod 216 is, in one embodiment, a rigid structure which is resistant to bending. Due to the straight, axial structure, i.e., the lack of an arcuate shape, of EFD 200, an internal support structure, such as EFD support 160 (FIGS. 5-6) described above, is not necessarily required to prevent inward expansion of radially expandable fingers 202 upon deformation of EFD 200. EFD rod 216 can be threaded into threaded recess 210 of EFD 200 and EFD actuator 1000 (FIGS. 17-18) or EFD actuator 1050 (FIGS. 19-20), described below, or another similar actuation device, may be used to deform radially expandable fingers 202 to the position shown in FIGS. 8-9. In an alternative embodiment, an internal support, similar to EFD support 160 (FIGS. 5-6), described above, is included inside EFD 200 and threaded end 217 of EFD rod 216 is threaded into a threaded recess disposed in the lateral end of the internal support.

Referring now to FIG. 10, in another alternative embodiment, EFD 250 is shown which, except as described below, is substantially similar in structure and operation to EFD 200 (FIGS. 7-9) described above. EFD 250 includes body 254, radially expandable fingers or elements 252, medial end 256, and lateral end 262. EFD 250 is sized smaller than EFD 200 to accommodate applications requiring the use of smaller fixation devices.

Referring now to FIG. 11, in yet another alternative embodiment, EFD 300 is shown which, except as described below, is substantially similar in structure and operation to EFD 250 (FIG. 10) described above. EFD 300 includes body 304, radially expandable fingers or elements 302, medial end 306, and lateral end 312. Each radially expandable finger 302 is defined between outer circumferential grooves 330 and between respective pairs of longitudinal cuts 340. Longitudinal cuts 340 are parallel to central axis 301. Outer circumferential grooves 330 and inner circumferential groove 332 create hinge points for radially expandable fingers 302 and facilitate deformation thereof by defining localized areas of reduced wall thickness. The central portion of each radially expandable finger 302 may include small central cutouts 329 on one or opposing sides thereof which further facilitate deformation of radially expandable fingers 302 by defining a localized area of reduced thickness which provides a bending or hinge point. Lateral end 312 may include pedicle screw head 314 with aperture 315. Aperture 315 is sized to accept a fixation rod or cord used in spinal fixation surgery. EFD 300 may be used in Zimmer, Inc.'s DynesysE Dynamic Stabilization System, for example, wherein EFD 300 functions as the pedicle screw and aperture 315 receives a flexible cord therethrough to provide dynamic stabilization. In such an application, EFD 300 is anchored in the vertebral body through the pedicle and aperture 315 remains outside of the pedicle to provide for passage of the flexible cord. EFD 300 may have an elliptical or oval cross-section to advantageously facilitate placement of EFD 300 in a bone structure, such as a pedicle.

EFD 300 also advantageously removes the requirement of precisely orienting a conventional pedicle screw. With a conventional pedicle screw, the orientation of aperture 315 is critical for aligning with the connecting rod or cord. It is difficult to obtain the correct orientation of aperture 315 without unthreading or rethreading the pedicle screw in the pedicle. EFD 300 eliminates these problems because EFD 300 may be inserted into the bone and aperture 315 may be aligned prior to deformation of radially expandable fingers 302. EFD 300 may be correspondingly adjusted and then deformed to provide fixation in the pedicle with aperture 315 correctly aligned to receive the flexible cord or fixation rod. Although described above as used with EFD 300, pedicle screw head 314 may be similarly disposed on any of the EFDs described throughout this application.

Referring now to FIGS. 12-16, in yet still another embodiment, EFD 350 is shown which, except as described below, is substantially similar in structure and operation to EFD 200 (FIGS. 7-9) described above. EFD 350 includes body 354, radially expandable fingers or elements 352, medial end 356, and lateral end 362. Each radially expandable finger 352 is defined by circumferential groove 380, medial end 356, and respective pairs of longitudinal cuts 390. Longitudinal cuts 390 in EFD 350 extend from circumferential groove 380 to medial end 356. Longitudinal cuts 390 are not bounded by any material on medial end 356 which allows the deformation of radially expandable fingers 352 as shown in FIG. 12 and described below. Longitudinal cuts 390 are oriented parallel to central axis 351 prior to deformation of fingers 352.

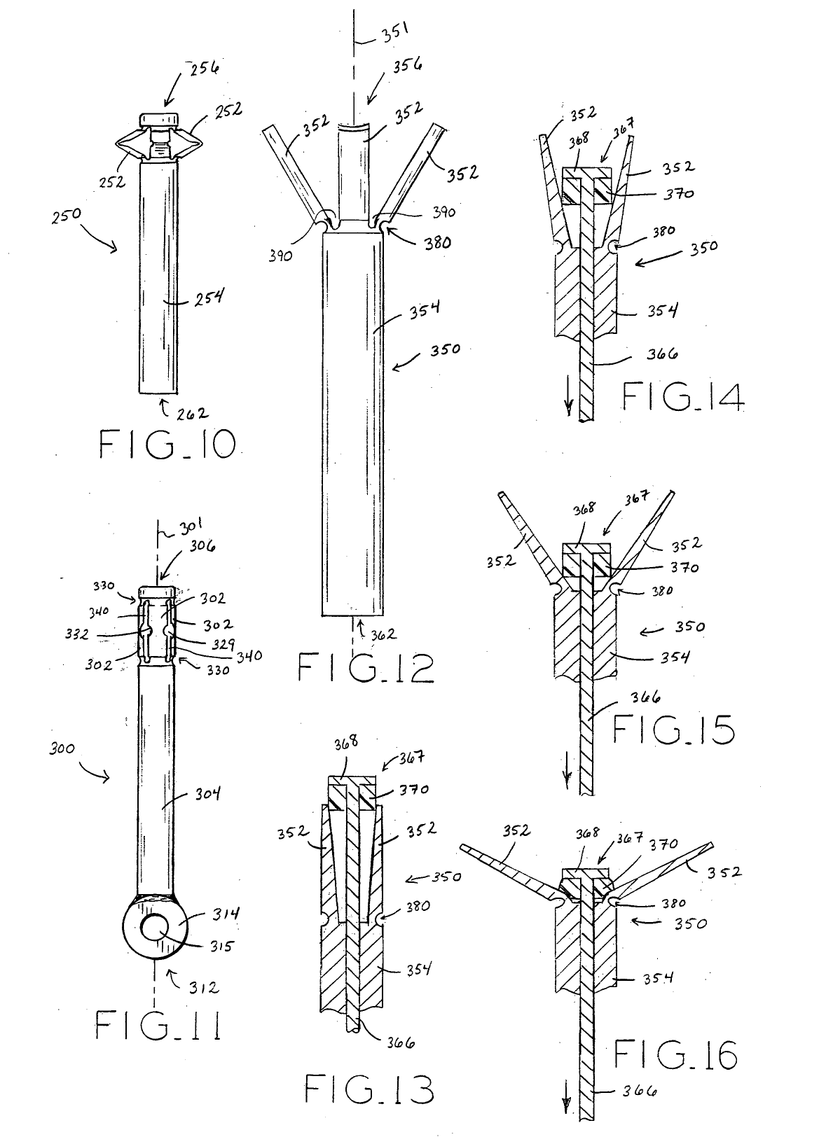

Referring to FIGS. 13-16, to effect deformation of radially expandable fingers 352, deployment device 367 may be employed. Deployment device 367 includes rod 366 with plunger 368 positioned on a medial end thereof. Bead 370 is positioned around rod 366 proximate plunger 368. Deployment device 367 is inserted into EFD 350 before EFD 350 is inserted into a patient. The lateral end of rod 366 is inserted through medial end 356 of EFD 350 until bead 370 is in abutting engagement with radially expandable fingers 352, as shown in FIG. 13. The lateral end of rod 366 may be coupled with a device to effect pulling of deployment device 367, such as EFD actuator 1000 (FIGS. 17-18) or EFD actuator 1050 (FIGS. 19-20), for example. Upon pulling of rod 366 in a lateral axial direction, plunger 368 forces bead 370 into contact with radially expandable fingers 352. After slight pulling of rod 366, bead 370 causes radially expandable fingers 352 to deform slightly, as shown in FIG. 14. Upon more pulling of rod 366, bead 370 causes greater deformation of radially expandable fingers 352 until plunger 368 cannot move further without deforming bead 370, as shown in FIG. 15. Once the deformation has reached this stage, the surgeon may pull further to effect deformation of bead 370, as shown in FIG. 16. Bead 370 may advantageously be formed of a deformable plastic or polymer material which deforms under compressive forces. Alternatively, bead 370 could be formed of a metal alloy which readily deforms under compressive forces, for example, nitinol. Once bead 370 deforms, rod 366 can be pulled slightly further to provide even further deformation of radially expandable fingers 352, as shown in FIG. 16.

Advantageously, the diameter of plunger 368 of deployment device 367 is the same or smaller than the diameter of EFD 350 to avoid making an access hole in a patient larger than the diameter of EFD 350. However, the access hole could be made slightly larger to accommodate a larger diameter plunger 368 to effect greater deformation of radially expandable fingers 352, i.e., the larger access hole would be compensated for by the greater deformation of radially expandable fingers 352 due to the larger diameter of plunger 368. Furthermore, bead 370 may be removed and replaced with a larger plunger 368.

2. Small Anchor-Type Expandable Fixation Devices

The EFDs described in this section are generally characterized by multiple sets of shorter longitudinal cuts parallel to the central axis or concentric to the central curve of the part. Each EFD contains multiple levels of expansion. The EFDs are capable of expanding to a size significantly greater than their original geometry, e.g., 75%-100% increase in effective diameter depending on the application.

Referring now to FIGS. 21-22, in one embodiment, EFD 400 is shown. EFD 400 includes body 402, central axis 401, medial end 420, lateral end 422, and multiple sets of longitudinal apertures 403, 405, 407, and 409 with corresponding intermediate material regions 404, 406, 408, and 410, respectively. The longitudinal apertures are generally disposed around a circumference of EFD 400 and are generally aligned parallel with central axis 401.

The first set of longitudinal apertures includes four apertures 403 equally spaced around the circumference of EFD 400 near medial end 420. The next set of longitudinal apertures includes four apertures 405 equally spaced around the circumference of EFD 400 proximate apertures 403 and staggered with respect to apertures 403 such that each aperture 405 is essentially longitudinally aligned with each intermediate material region 404. The next set of longitudinal apertures includes four apertures 407 equally spaced around the circumference of EFD 400 proximate apertures 405 and are substantially longitudinally aligned with apertures 403. The final set of longitudinal apertures includes four apertures 409 equally spaced around the circumference of EFD 400 proximate apertures 407 and are substantially longitudinally aligned with apertures 405. The longitudinal distance L between each set of longitudinal apertures may vary depending on the particular application, and, in one embodiment, a minimum value for distance L may be between approximately 25% and 75% of the longitudinal length of apertures 403, 405, 407, 409. Distance L may be determined based upon a combination of factors including required expanding diameter of EFD 400 and the overall initial part length, both of which are primarily dictated by the desired application. The maximum value for distance L typically is between 75% and 150% of the longitudinal length of apertures 403, 405, 407, 409, but may be varied depending on the particular application.

Each longitudinal aperture 403 has width 403W, each longitudinal aperture 405 has width 405W, each longitudinal aperture 407 has width 407W, and each longitudinal aperture 409 has width 409W. Width 403W is larger than width 405W, which is larger than width 407W, which is larger than width 409W. The increasing widths of the longitudinal apertures towards medial end 420, in turn, causes the widths of the intermediate material regions between each aperture, e.g., the width between aperture 403 and an adjacent aperture 403 to be less than the width between aperture 405 and an adjacent aperture 405, and so on towards lateral end 422. Advantageously, the greater the width of the aperture, the more readily the intermediate material region will deform. For example, upon a deformation force being applied to EFD 400 with EFD actuator 1000 (FIGS. 17-18) or EFD actuator 1050 (FIGS. 19-20), described below, or another similar actuator device, intermediate material region 404 will deform first, followed by deformation of intermediate material region 406, followed by deformation of intermediate material region 408, and, finally, deformation of intermediate material region 410. Such an arrangement ensures that all intermediate material regions will deform, as shown in FIG. 22, to facilitate a stronger engagement with bone stock in a patient. Widths 403W, 405W, 407W, 409W and the corresponding widths for the intermediate material regions may be varied depending on the desired result and/or application, for example, the width of intermediate material region 404 may be approximately equal to the width of EFD 400, or, alternatively, may be approximately equal to 403W. Furthermore, although only four sets of apertures are shown in EFD 400, additional sets of apertures may be added to EFD 400. The fixation achieved by the deformation of EFD 400 can be varied by adjusting the longitudinal lengths of the intermediate material regions, i.e., longer intermediate material regions will provide greater expansion and fixation. EFD 400 will essentially expand to a diameter which is equal to its maximum effective diameter plus the length of the largest intermediate material region because the longitudinal apertures are disposed in 90° steps around the circumference of EFD 400; therefore each intermediate material region opposes an identical region of intermediate material. Each region of intermediate material will expand to, at the most, half of the longitudinal distance of each region.

Advantageously, EFD 400 may be constructed from material having a wall thickness between about 0.015 in. and about 0.025 in. or preferably about 0.020 in., but other thicknesses may be used depending on the desired bending input load and fatigue life. An EFD support, similar to EFD support 160 (FIGS. 5-6) described above, may be inserted within EFD 400 to provide enhanced structural support. The minimal wall thickness necessary for EFD 400 advantageously decreases manufacturing and material costs, while the inclusion of an EFD support maintains the structural integrity of the device.

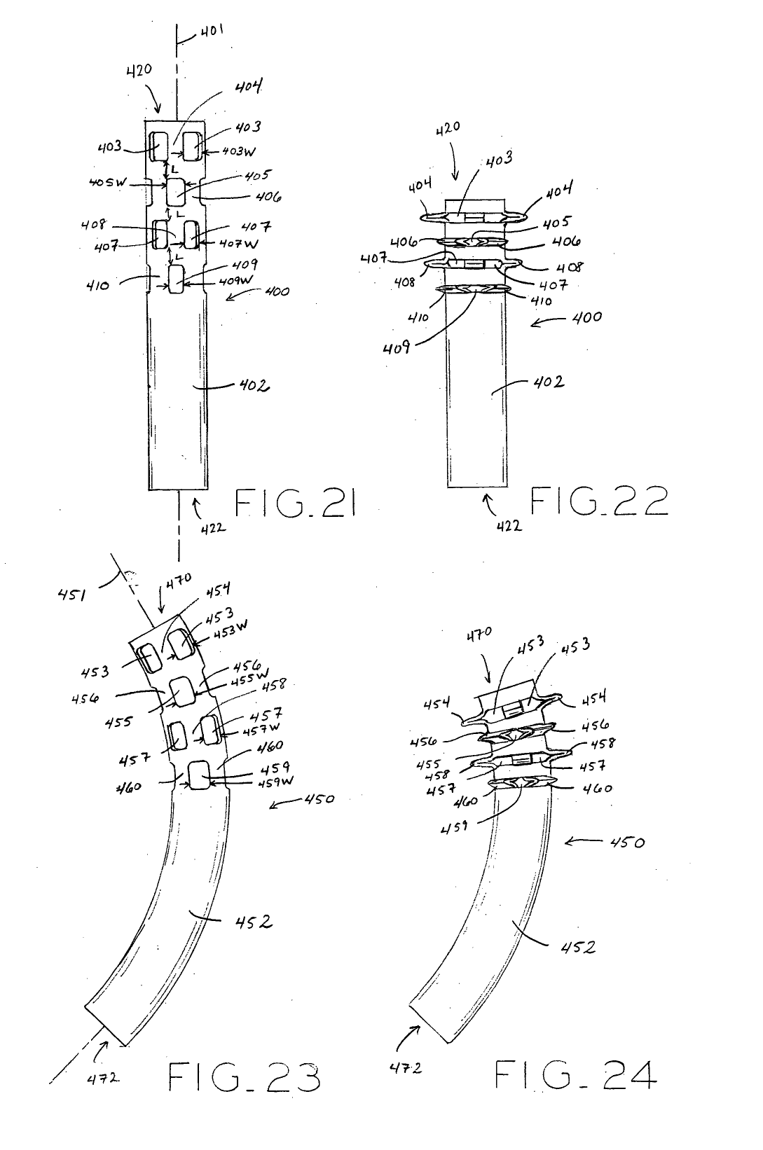

Referring now to FIGS. 23-24, in another embodiment, EFD 450 is shown which, except as described below, is substantially similar in structure and operation to EFD 400 (FIGS. 21-22) described above. EFD 450 may have an arcuate configuration having central axis 451 and body 452, longitudinal apertures 453, 455, 457, 459, and intermediate material regions 454, 456, 458, 460 which are deformable similar to regions 404, 406, 408, and 410 (FIG. 22) described above.

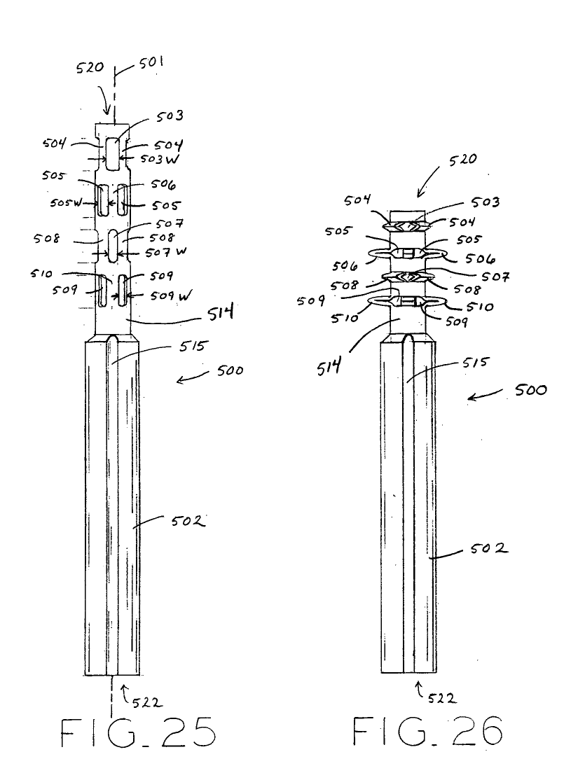

Referring now to FIGS. 25-26, in yet another embodiment, EFD 500 is shown which, except as described below, is substantially similar in structure and operation to EFD 400 (FIGS. 21-22) described above. EFD 500 includes body 502 and deformable body portion 514 having longitudinal apertures 503, 505, 507, 509, and intermediate material regions 504, 506, 508, 510. Body 502 includes keyway or groove 515 longitudinally extending from lateral end 522 to deformable body portion 514. Deformable body portion 514 has a smaller outside diameter than the outside diameter of body 502 to facilitate entry through a hip plate (not shown). For example, a hip plate may include a key disposed in the lag screw aperture. The key is provided to mate with the hip or lag screw to prevent rotation of the hip screw once inserted into bone. Keyway 515 may mate with this key in the hip plate. Deformable body portion 514 has a smaller outside diameter such that, when EFD 500 is inserted through a hip plate, or any other orthopedic implant or instrument with a key configuration, deformable body portion 514 may traverse the aperture without impinging on the key structure. Furthermore, the smaller outside diameter of deformable body portion 514 facilitates easier deformation of intermediate material regions 504, 506, 508, and 510 because the material thickness of deformable body portion 514 is thinner than the material thickness of body 502. Although described above with reference to EFD 500, keyway 515 may be disposed on any of the EFDs described throughout this application in a similar manner.

3. Screw-Type Expandable Fixation Devices

The EFDs in this section are generally characterized by multiple rotationally indexed cuts arranged in a helical pattern. The cuts on the EFDs are designed such that, when deformed, the expansions form a substantially helical thread along the length of the EFD. The EFDs are capable of expanding up to approximately 100% in effective diameter. An advantage of the EFDs described in this section is that the EFDs may be removed from a patient by unthreading the EFD, thereby minimizing tissue damage upon removal. Additionally, the EFDs described in this section eliminate the need to “un-deform” the expanded fingers prior to removal.

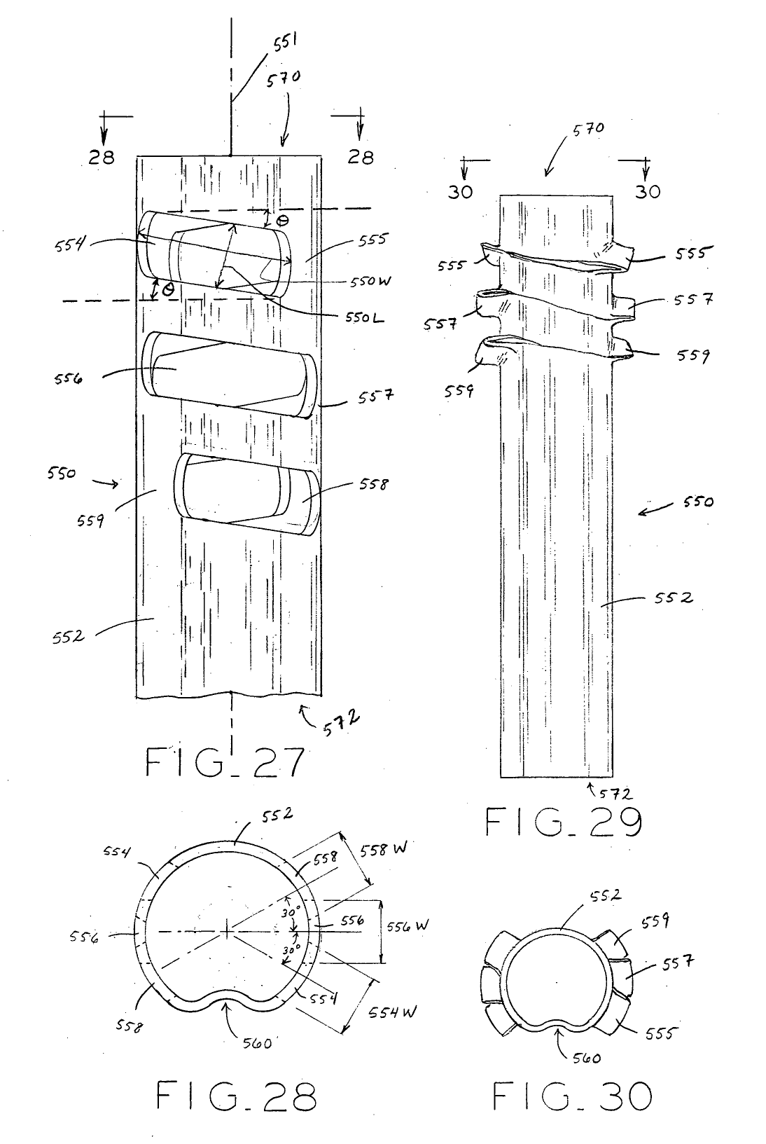

Referring now to FIGS. 27-30, EFD 550 is shown including body 552, slanted apertures 554, 556, 558, and intermediate material regions 555, 557, 559. EFD 550 has lateral end 572 and medial end 570. As shown in FIG. 28, slanted aperture 554 has width 554W, slanted aperture 556 has width 556W, and slanted aperture 558 has width 558W. Each slanted aperture is oriented 30° around the circumference of body 552, i.e., after slanted aperture 554 is formed, EFD 550 is rotated 30° clockwise (as viewed from medial end 570) and then an identical cut for slanted aperture 556 is made. Similarly, after slanted aperture 556 is formed, EFD 550 is rotated 30° clockwise and then an identical cut for slanted aperture 558 is formed. The slanted apertures may be formed or cut by any cutting or forming technique known in the art.

Body 552 may include keyway 560, similar to keyway 515 described above with respect to EFD 500 (FIGS. 25-26). In one embodiment, no slanted aperture is formed where keyway 560 is located on body 552, i.e., if EFD 550 is rotated and keyway 560 is to be cut through, EFD 550 is rotated further until the slanted aperture contains no part of keyway 560.

As shown in FIGS. 29-30, deformation of EFD 550 by any of the methods described herein causes intermediate material regions 555, 557, 559 to expand outwardly. The expansion of intermediate material regions 555, 557, 559 forms protrusions on EFD 550 which, taken together, form a non-continuous helical thread. Alternatively, by varying the ranges of the apertures and the intermediate material regions, the protrusions on EFD 550 may form a more complete helical thread. The thread facilitates securement of EFD 550 in a bone structure and also advantageously facilitates removal of EFD 550 from a bone structure while minimizing potential tissue damage. The thread provides all of the advantages of a traditional hip screw while advantageously having a smaller diameter upon initial insertion into a patient.

As shown in FIG. 27, slanted apertures 554, 556, 558 are formed in a generally rectangular shape with rounded corners and ends and the apertures are oriented at an angle θ with respect to a line perpendicular to central axis 551. Angle θ may be between 10° and 45°, or, preferably between 10° and 30°, but the angle may be varied based on the desired behavior of EFD 550. Each aperture has dimension 550L which may be between 0.045 in. and 0.250 in., but this dimension may be varied depending on the desired expanded diameter of EFD 550. Dimension 550W is a result of dimension 550L and the depth of apertures 554, 556, 558 relative to the outer surface of EFD 550.

Advantageously, EFD 550 may be constructed from material having a wall thickness between about 0.015 in. and about 0.025 in., or preferably about 0.020 in., but other thicknesses may be used depending on the desired bending input load and fatigue life. An EFD support, similar to EFD support 160 (FIGS. 5-6) described above, may be inserted within EFD 550 to provide enhanced structural support. The minimal wall thickness necessary for EFD 550 advantageously decreases manufacturing and material costs, while the inclusion of an EFD support maintains the structural integrity of the device.

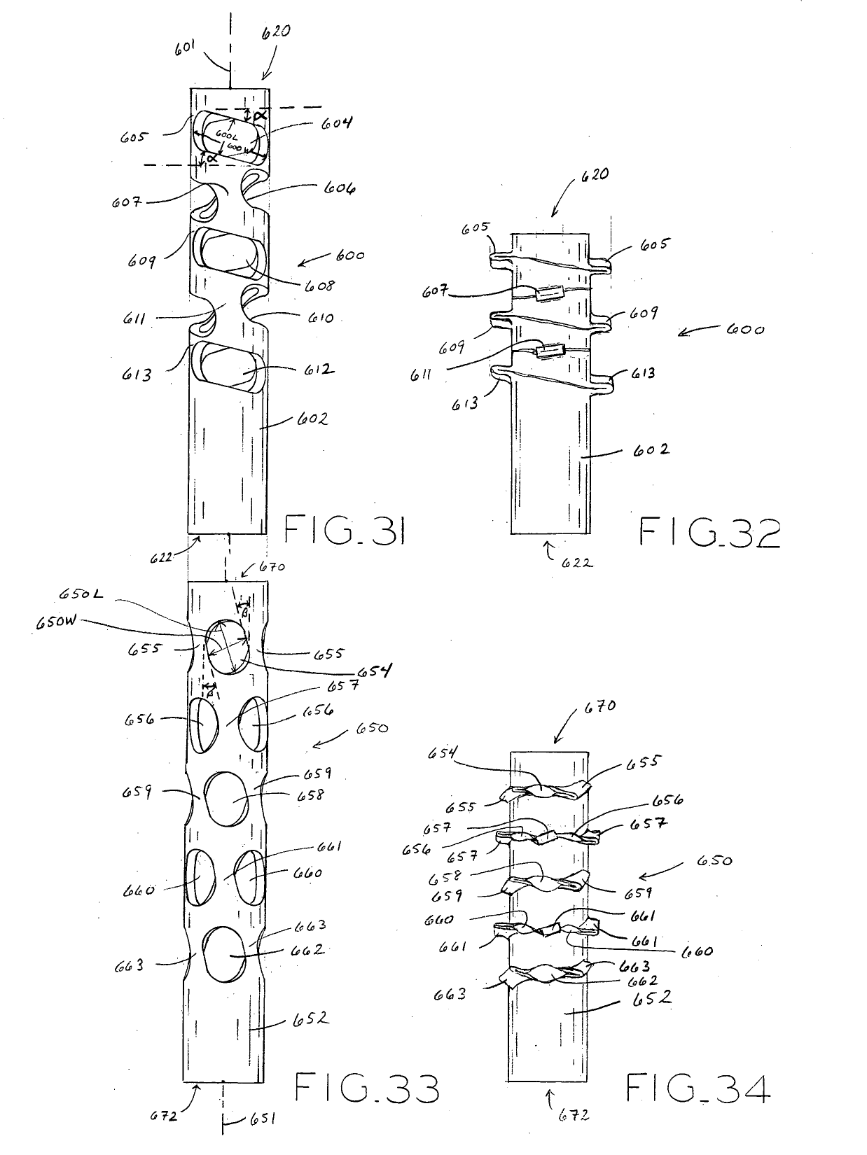

Referring now to FIGS. 31-32, in another embodiment, EFD 600 is shown which, except as described below, is substantially similar in structure and operation to EFD 550 (FIGS. 27-30) described above. EFD 600 includes body 602, slanted apertures 604, 606, 608, 610, 612, intermediate material regions 605, 607, 609, 611, 613, lateral end 622, and medial end 620. Each slanted aperture is oriented 90° around the circumference of body 602, i.e., after slanted aperture 604 is formed, EFD 600 is rotated 90° clockwise (as viewed from medial end 620) and then an identical cut for slanted aperture 606 is made. Similarly, after slanted aperture 606 is formed, EFD 600 is rotated 90° clockwise and then an identical cut for slanted aperture 608 is formed. Similarly, after slanted aperture 608 is formed, EFD 600 is rotated 90° clockwise and then an identical cut for slanted aperture 610 is formed after which EFD 600 is rotated 90° clockwise and then an identical cut for slanted aperture 612 is formed.

As shown in FIG. 32, deformation of EFD 600 by any of the methods described herein causes intermediate material regions 605, 607, 609, 611, 613 to expand outwardly. The expansion of intermediate material regions 605, 607, 609, 611, 613 forms protrusions on EFD 600 which, taken together, form a non-continuous helical thread. Alternatively, by varying the ranges of the apertures and the intermediate material regions, the protrusions on EFD 600 may form a more complete helical thread. The thread facilitates securement of EFD 600 in a bone structure and also advantageously facilitates removal of EFD 600 from a bone structure while minimizing potential tissue damage. The thread forms a double-lead thread for EFD 600 to provide for enhanced fixation. The double-lead thread provides more threads over the length of EFD 600 but advantageously requires fewer rotations to thread EFD 600 out of a bone.

As shown in FIG. 31, slanted apertures 604, 606, 608, 610, 612 are formed in a generally rectangular shape with rounded corners and ends and the apertures are oriented at an angle α with respect to a line perpendicular to central axis 601. Angle α may be between 10° and 45°, or, preferably between 10° and 30°, but the angle may be varied based on the desired behavior of EFD 550. Each aperture has dimension 600L which may be between 0.045 in. and 0.250 in., but this dimension may be varied depending on the desired expanded diameter of EFD 600. Dimension 600W is a result of dimension 600L and the depth of apertures 604, 606, 608, 610, 612 relative to the outer surface of EFD 600.

Referring now to FIGS. 33-34, in yet another embodiment, EFD 650 is shown which, except as described below, is substantially similar in structure and operation to EFD 550 (FIGS. 27-30) described above. EFD 650 includes body 652, apertures 654, 656, 658, 660, 662, intermediate material regions 655, 657, 659, 661, 663, lateral end 672, and medial end 670. Each aperture is oriented 45° around the circumference of body 652, i.e., after aperture 654 is formed, EFD 650 is rotated 45° clockwise (as viewed from medial end 670) and then an identical cut for aperture 656 is made. Similarly, after aperture 656 is formed, EFD 650 is rotated 45° clockwise and then an identical cut for aperture 658 is formed. Similarly, after aperture 658 is formed, EFD 650 is rotated 45° clockwise and then an identical cut for aperture 660 is formed after which EFD 650 is rotated 45° clockwise and then an identical cut for aperture 662 is formed.

As shown in FIG. 34, deformation of EFD 650 by any of the methods described herein causes intermediate material regions 655, 657, 659, 661, 663 to expand outwardly. The expansion of intermediate material regions 655, 657, 659, 661, 663 forms protrusions on EFD 650 which, taken together, form a non-continuous helical thread. Alternatively, by varying the ranges of the apertures and the intermediate material regions, the protrusions on EFD 650 may form a more complete helical thread. The thread facilitates securement of EFD 650 in a bone structure and also advantageously facilitates removal of EFD 650 from a bone structure while minimizing potential tissue damage. The thread forms a quadruple-lead thread for EFD 650 to provide for enhanced fixation. The quadruple-lead thread provides more threads over the length of EFD 650 but advantageously requires fewer rotations to thread EFD 650 out of a bone.

As shown in FIG. 33, apertures 654, 656, 658, 660, 662 are formed in a generally rectangular shape with rounded corners and ends and the apertures are oriented at an angle β with respect to a line parallel to central axis 651. Angle β may be between 45° and 90°, or, preferably between 55° and 75°, but the angle may be varied depending on the desired helix angle. Each aperture has dimensions 650L and 650W which may be between 0.15 in. and 0.20 in., or, preferably, 0.175 in. for dimension 650L and between 0.10 in. and 0.15 in., or, preferably, 0.125 in. for dimension 650W.

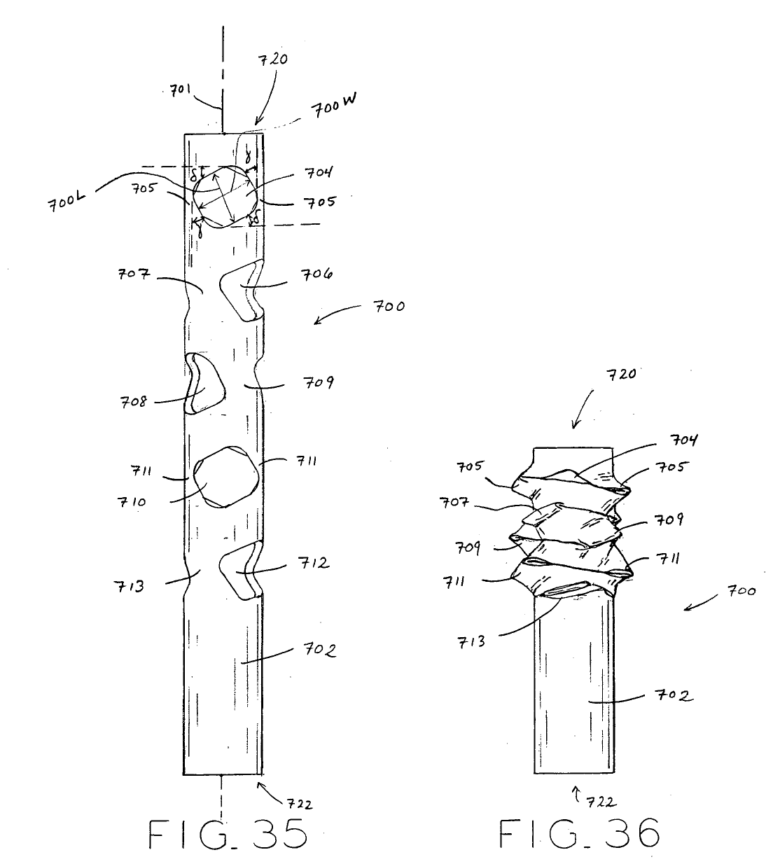

Referring now to FIGS. 35-36, in a still further embodiment, EFD 700 is shown which, except as described below, is substantially similar in structure and operation to EFD 550 (FIGS. 27-30) described above. EFD 700 includes body 702, apertures 704, 706, 708, 710, 712, intermediate material regions 705, 707, 709, 711, 713, lateral end 722, and medial end 720. Each aperture is oriented 60° around the circumference of body 702, i.e., after aperture 704 is formed, EFD 700 is rotated 60° clockwise (as viewed from medial end 720) and then an identical cut for aperture 706 is made. Similarly, after aperture 706 is formed, EFD 700 is rotated 60° clockwise and then an identical cut for aperture 708 is formed. Similarly, after aperture 708 is formed, EFD 700 is rotated 60° clockwise and then an identical cut for aperture 710 is formed after which EFD 700 is rotated 60° clockwise and then an identical cut for aperture 712 is formed.

As shown in FIG. 36, deformation of EFD 700 by any of the methods described herein causes intermediate material regions 705, 707, 709, 711, 713 to expand outwardly. The expansion of intermediate material regions 705, 707, 709, 711, 713 forms protrusions on EFD 700 which, taken together, form a non-continuous helical thread. Alternatively, by varying the ranges of the apertures and the intermediate material regions, the protrusions on EFD 700 may form a more complete helical thread. The thread facilitates securement of EFD 700 in a bone structure and also advantageously facilitates removal of EFD 700 from a bone structure while minimizing potential tissue damage. The thread forms a triple-lead thread for EFD 700 to provide for enhanced fixation in a bone.

As shown in FIG. 35, apertures 704, 706, 708, 710, 712 are formed in a generally square shape with rounded corners and the apertures are oriented at an angle γ with respect to a line parallel to central axis 701. Angle γ may be between 10° and 40°, or, preferably 26.68°. Apertures 704, 706, 708, 710, 712 are also oriented at an angle δ with respect to a line perpendicular to central axis 701. Angle δ may be between 10° and 40°, or, preferably 26.68°. When apertures 704, 706, 708, 710, 712 are generally square-shaped, angle γ is equal to angle δ. When apertures 704, 706, 708, 710, 712 are not square-shaped, this no longer holds true and angle γ is not equal to angle δ. Each aperture has dimensions 700L and 700W which may be approximately 0.188 in. for dimension 700W and 0.175 in. for dimension 700L. Dimensions 700W and 700L are controlled by the inside and outside diameters of EFD 700, the values of angle γ and angle δ, and the desired expanded diameter of EFD 700.

B. EFD Deployment Devices

Referring now to FIGS. 17-18, to effect deformation of the radially expandable fingers of the EFDs of the present invention, an exemplary EFD actuator 1000 is shown. Although described below with reference to EFD 100, EFD actuator 1000 may be used to effect deformation of any EFD described above. EFD actuator 1000 may be used to apply compression to EFD 100 and cause deformation of radially expandable fingers 102. EFD actuator 1000 includes body 1002 with handle 1004, intermediate shaft 1006, guide 1008, wrench 1010, linear screw 1012, nut 1014, cable anchor 1016, and compression device 1020 including cable 116, anchor 110, medial spacer 118, and lateral spacer 120, described above (FIGS. 3-4). Body 1002 includes counterbore 1003 and a throughbore (not shown) shaped to axially receive linear screw 1012. The throughbore in body 1002 is large enough to receive linear screw 1012 but prevents entrance by intermediate shaft 1006 into the interior of body 1002. Thus, the lateral end of intermediate shaft 1006 butts against the medial end of body 1002. Linear screw 1012 may be an acme screw having at least one flat 1013 longitudinally extending along the length of linear screw 1012. The throughbore in body 1002 correspondingly includes at least one flat (not shown) so that linear screw 1012 may be axially received into the throughbore while preventing screw 1012 from rotating within body 1002.

Nut 1014 includes external teeth 1015 which may be engaged by corresponding internal teeth 1011 of wrench 1010 for rotational actuation of nut 1014 by wrench 1010. Nut 1014 also includes internal thread 1017 which mates with threads 1018 of linear screw 1012. In operation, rotation of wrench 1010 on nut 1014 axially translates linear screw 1012 relative to body 1002. Axial translation rather than rotation is provided because of the matching of flat 1013 of linear screw 1012 with the internal flat of the throughbore in body 1002. Linear screw 1012 may be cannulated by throughbore 1019 which receives cable 116 therethrough. Intermediate shaft 1006 and guide 1008 are also cannulated to receive cable 116 through throughbore 1007. Guide 1008 may include a substantially elliptical, or, alternatively, a circular or polygonal, cross-section. Guide 1008 may also be provided with cuts 1009 to provide flexibility in a single plane. Alternatively, guide 1008 may be a rigid tube with limited flexibility.

Anchor 110 may be coupled to cable 116 in order to withstand a tension of at least approximately 454 kg (1,000 pounds). Lateral end 125 of cable 116 is coupled to threaded shaft 126. Threaded shaft 126 may be received through throughbore 1007 of guide 1008, the throughbore of body 1002, internal teeth 1011 of wrench 1010, throughbore 1019 of linear screw 1012, internal thread 1017 of nut 1014, and engaged with internal thread 1021 of cable anchor 1016. Once threaded shaft 126 is engaged with internal thread 1021 of cable anchor 1016 and engagement ears 136 of anchor 110 are engaged with apertures 108 of EFD 100, wrench 1010 may be rotated. Rotation of wrench 1010 causes rotation of nut 1014, which, in turn, causes axial movement of linear screw 1012 due to the engagement of threads 1017 of nut 1014 and threads 1018 of screw 1012 as well as the engagement of flats 1013 of screw 1012 and the matching flats of the throughbore in body 1002. Lateral axial movement of linear screw 1012 displaces cable anchor 1016 and therefore cable 116 laterally relative to guide 1008. Lateral displacement of cable 116 forces lateral end 112 of EFD 100 against guide 1008. Upon further lateral displacement of cable 116, anchor 110 pulls medial end 106 of EFD 100 toward lateral end 112 of EFD 100, thereby deforming radially expandable fingers 102 to an expanded state, as shown in FIG. 4.

Upon desired deformation of EFD 100, wrench 1010 may be rotated in the opposite direction to loosen the tension on cable 116. EFD actuator 1000 may then be removed from cable 116 by unthreading threaded shaft 126 from internal thread 1021 and sliding cable 116 out of EFD actuator 1000. Cable 116 may be removed from EFD 100 by turning cable 116 clockwise or counterclockwise 90° to release anchor 110 from engagement with apertures 108 of EFD 100 and then sliding cable 116 out of EFD 100. In one embodiment, EFD actuator 1000 is capable of compressing EFD 100 to a load of at least about 454 kg (1,000 lbs.) or at least about twice the deformation force of EFD 100.

Referring now to FIGS. 19-20, to effect deformation of the radially expandable fingers of the EFDs of the present invention, an alternative embodiment EFD actuator 1050 may be used. Although described below with reference to EFD 150, EFD actuator 1050 may be used to effect deformation of any EFD described above. EFD actuator 1050 may be used to apply compression to EFD 150 and cause deformation of radially expandable fingers 152. EFD actuator 1050 includes body 1052 with handle 1054, wrench 1060, linear screw 1064, and guide 1058. Body 1052 includes a plurality of threaded handle bores 1056 into which handle 1054 may be screwed. The plurality of bores 1056 provides a surgeon with several options for the location of handle 1054 depending on the anatomy of the patient. Body 1052 includes throughbore 1055 shaped to axially receive linear screw 1064 therein. Throughbore 1055 is large enough to receive linear screw 1064 but prevents entrance by guide 1058 into the interior of body 1052. Thus, the lateral end of guide 1058 abuts the medial end of body 1052. Linear screw 1064 may be an acme screw having at least one flat 1065 longitudinally extending along the length of linear screw 1064. Throughbore 1055 in body 1052 correspondingly includes at least one flat (not shown) so that linear screw 1064 may be axially received into throughbore 1055 while preventing linear screw 1064 from rotating within body 1052.

EFD actuator 1050 also includes actuator nut 1066 having internal threads 1069 corresponding to threads 1070 of screw 1064. Nut 1066 also includes polygonal section 1067 on at least a portion of its exterior to mate with wrench 1060 for rotational actuation of nut 1066. Wrench 1060 includes handle 1061 and body 1062 configured to mate with nut 1066. In operation, rotation of wrench 1060 on nut 1066 axially translates screw 1064 relative to body 1052. Axial rather than rotational translation is provided because of the matching of flat 1065 of linear screw 1064 with the internal flat of throughbore 1055 in body 1052.

To assemble EFD actuator 1050 to an EFD, for example, EFD 150 (FIGS. 5-6), threaded end 167 of EFD cable 166 is threaded into the recess in the lateral end of EFD support 160, or, alternatively, directly into a threaded recess similar to threaded recess 210 (FIG. 9), described above. Lateral end 168 of EFD cable 166 is then slid through throughbore 1057 in shaft 1058, throughbore 1055 in body 1052, and throughbore 1071 in linear screw 1064. Once lateral end 168 extends laterally beyond linear screw 1064, EFD cable 166 is moved into engagement with a counterbore (not shown) in the lateral end of linear screw 1064 which has a finite depth and a diameter approximately equal to the diameter of throughbore 1071. Secondary throughbore 1072 and the counterbore are sized to permit movement of EFD cable 166 through linear screw 1064 but prevent movement of lateral end 168 therethrough, essentially locking lateral end 168 to linear screw 1064 and preventing lateral end 168 from medially moving with respect to linear screw 1064 when locked therein.

In an exemplary embodiment, EFD cable 166 has a length which, when lateral end 168 is secured in linear screw 1064 and threaded end 167 of EFD cable 166 is secured in EFD support 160, lateral end 162 of EFD 150 is in abutting relationship with the medial end of guide 1058. Guide 1058 may include cuts 1059 to facilitate flexing of guide 1058 depending on the particular application. Alternatively, guide 1058 may be a rigid tube with limited flexibility. Guide 1058 may have an elliptical, or, alternatively, a circular or polygonal, cross-sectional shape. Additionally, EFD cable 166 may be a flexible structure, or, alternatively, a rigid rod, depending on the desired application.

Once EFD actuator 1050 is assembled to EFD 150, wrench 1060 is rotated and turns actuator nut 1066. Turning of actuator nut 1066 causes linear screw 1064 to axially translate in a lateral direction due to the engagement of threads 1069 of actuator nut 1066 and threads 1070 of linear screw 1064 as well as the engagement of flat 1065 of linear screw 1064 with the flat in throughbore 1055. Lateral axial translation of linear screw 1064 consequently translates EFD cable 166 in a lateral axial direction. Translation of EFD cable 166 forces medial end 156 of EFD 150 to be pulled toward lateral end 162 because of the connection of EFD support 160 with EFD 150 via transverse pin 164. Guide 1058 is prevented from moving laterally by the abutting engagement of the lateral end of guide 1058 with the medial end of body 1052 and the medial end of guide 1058 prevents lateral movement of body 154 of EFD 150. Consequently, the compressive forces cause the deformation of radially expandable fingers 154, as shown in FIG. 6.

Once fingers 154 are adequately deployed, wrench 1060 is rotated in an opposite direction which turns actuator nut 1066 to release tension on EFD cable 166. Lateral end 168 of EFD cable 166 may then be slid out of engagement with secondary throughbore 1072 and slid out of linear screw 1064 via throughbore 1071. Body 1052 and guide 1058 may then be slid off of EFD cable 166 prior to or subsequent to threaded end 167 of EFD cable 166 unthreading from the threaded recess in the lateral end of EFD support 160.

In one embodiment, EFD actuator 1050 is capable of compressing EFD 150 to a load of at least about 454 kg (1,000 lbs.) or at least about twice the deformation force of EFD 150.

While this invention has been described as having exemplary designs, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. An expandable fixation device having a first end and a second end for use in orthopedic surgery for anchoring within an anatomical structure, comprising:
a body having a central axis and including at least one expandable element, each said expandable element defined between a pair of openings in said body, wherein upon compressive loading of said body, each said expandable element expands radially with respect to said central axis to anchor the device within the anatomical structure.

2. The expandable fixation device of claim 1, wherein said openings comprise slots formed in said body.

3. The expandable fixation device of claim 1, wherein said openings comprise line cuts formed in said body.

4. The expandable fixation device of claim 1, wherein said openings are substantially longitudinally oriented, said longitudinal openings substantially parallel to said central axis.

5. The expandable fixation device of claim 1, wherein said openings are longitudinally elongated with respect to said central axis.

6. The expandable fixation device of claim 1, wherein said openings are elongated in a direction transverse to said central axis.

7. The expandable fixation device of claim 1, wherein said openings have a width and a length, said width less than said length.

8. The expandable fixation device of claim 1, wherein said openings have a width and a length, said length less than said width.

9. The expandable fixation device of claim 1, wherein said openings comprise a plurality of sets of openings circumferentially formed around said body, said plurality of sets including at least a first set and a second set of openings, said first set substantially longitudinally misaligned with respect to said second set.

10. The expandable fixation device of claim 1, wherein each said expandable element is further defined by at least one circumferential groove extending around said body.

11. The expandable fixation device of claim 1, wherein the device is curved.

12. The expandable fixation device of claim 1, further comprising a support structure at least partially disposed within said body.

13. The expandable fixation device of claim 1, further comprising a fixation structure disposed on the second end of the fixation device, said fixation structure including an aperture for receiving a fixation support element.

14. The expandable fixation device of claim 1, wherein said body includes a first portion and a second portion, said first portion including said at least one expandable element, said second portion having an outside diameter larger than an outside diameter of said first portion, said second portion including alignment structure extending from said first portion to the second end of the device, said alignment structure disposed on an exterior surface of said second portion.

15. An expandable fixation device having a first end and a second end for use in orthopedic surgery for anchoring within an anatomical structure, comprising:
a body having a central axis; and
at least one set of apertures formed in said body, said apertures circumferentially formed around said body and defining expandable portions between said apertures, at least some of said expandable portions axially and circumferentially staggered around said body, wherein, upon compressive loading of said body, said expandable portions radially expand and, taken together, form at least a portion of a helical thread.

16. The expandable fixation device of claim 15, wherein said apertures have a width and a length, said width less than said length.

17. The expandable fixation device of claim 15, wherein said apertures have a width and a length, said length less than said width.

18. The expandable fixation device of claim 15, wherein said apertures comprise a plurality of sets of apertures circumferentially formed around said body, said plurality of sets including at least a first set and a second set of apertures, said first set substantially longitudinally misaligned with respect to said second set.

19. The expandable fixation device of claim 15, further comprising a fixation structure disposed on the second end of the fixation device, said fixation structure including an aperture for receiving a fixation support element.

20. The expandable fixation device of claim 15, wherein said body includes a first portion and a second portion, said first portion including said at least one set of apertures, said second portion having an outside diameter larger than an outside diameter of said first portion, said second portion including alignment structure extending from said first portion to the second end of the device, said alignment structure disposed on an exterior surface of said second portion.

21. The expandable fixation device of claim 15, wherein the device is curved.

22. The expandable fixation device of claim 15, further comprising a support structure at least partially disposed within said body.
